(19) United States
(12) Patent Application Publication
(10) Pub. No.: US 2009/0185264 A1
(43) Pub. Date: Jul. 23, 2009

(54) Coupling optics for light transmission system

(75) Inventors: Brent D. Cameron, Waterville, OH (US); James A. Ohneck, South Russell, OH (US); Anthony J. Webb, Temperance, MI (US); Donald J. Styblo, Northfield Center, OH (US); Scott C. Molitor, Sylvania, OH (US)

Correspondence Address: James A. Hudak, Esq., Suite #304, 29425 Chagrin Blvd, Cleveland, OH, 44122, US

(73) Assignee: The University of Toledo and to Performance Improvement Corporation

(21) Appl. No.: 12/009,022

(22) Filed: Jan. 17, 2008

(51) Int. Cl. G02B 27/00; A61B 18/00

(52) U.S. Cl. 359/350; 606/3

(57) ABSTRACT

An infrared energy photo-biotherapy medical treatment device incorporating a Class IV laser light source is disclosed. The medical treatment device comprises a head assembly, a coupling device and the Class IV laser light source. A liquid light guide connects the head assembly to the coupling device and the Class IV laser light source is connected to the coupling device by a plurality of silica optical fibers. Optical lenses are incorporated in both the head assembly and the coupling device permitting the infrared energy photo-biotherapy medical treatment device to direct the infrared energy from the Class IV laser light source through the optical fibers, coupling device, liquid light guide and head assembly so as to penetrate into a living organism.

### TECHNICAL FIELD

The present invention relates, in general, to an infrared energy photo-biotherapy medical treatment device and, more particularly, to the optical coupling apparatus associated with an infrared energy photo-biotherapy medical treatment device that can produce a light that penetrates into a living organism.

### BACKGROUND ART

Infrared light at specific wavelengths has been proven to be effective in improving the healing process for some medical conditions that exist in both human beings and other warm-blooded animals. When applied to the skin, normal visible white light, visible red light, or invisible light and/or light with wavelengths from 300 nm to 1400 nm produces an increased thermal effect on the skin and the dermal layer to a depth of 1.0 mm. The relatively high level of energy being absorbed by the skin causes this effect. In numerous studies and clinical trials, infrared photo-biotherapy has been shown to provide significant therapeutic benefits. For example, tests conducted utilizing a scanning laser Doppler have indicated an increase in microcirculation from 400% to 3200% after one infrared light emitting diode treatment. Studies have also shown that human tissue exposed to infrared light from light emitting diodes grows at a rate of 150 to 200% faster than cells not stimulated by such light. Also, it has been shown that cells treated with infrared light exhibited a five-fold increase in growth phase specific DNA synthesis. Thus, the therapeutic benefits of infrared energy photo-biotherapy medical treatment are well documented.

One of the medical needs for which photo-biotherapy has proven to be useful is the treatment of decubitis ulcers. The typical medical approach to treat decubitis ulcers is to utilize cleansing agents, antiseptic agents, topical agents and/or dressings. While not typically utilized as a method of treatment, infrared energy photo-biotherapy has shown promise in the treatment of such ulcers. Such infrared energy photo-biotherapy has also shown promise in the medical treatment of numerous acute and chronic conditions including, but not limited to, carpal tunnel syndrome, back and neck pain, migraine headaches, wound healing, tendonitis, sprains, strains, repetitive stress injuries, arthritis and peripheral neuropathy.

Even though infrared energy photo-biotherapy medical treatment offers such promise, the use of such a therapy method is limited since most therapeutic devices that produce infrared energy are limited to an output power of less than 1.0 watt. Those infrared energy therapeutic medical treatment devices that produce significantly greater output power, e.g., 4.0 watts, are typically very expensive, large, cumbersome and difficult to transport.

In view of the foregoing disadvantages associated with presently available infrared energy therapeutic medical treatment devices, it has become desirable to develop a relatively low cost, portable, therapeutic medical treatment device utilizing a Class IV laser light source which produces a relatively high output power level.

### SUMMARY OF THE INVENTION

The present invention solves the problems associated with presently available infrared energy therapeutic medical treatment devices, and other problems, by providing a durable optical coupling apparatus that can be utilized by an infrared energy photo-biotherapy medical treatment device incorporating a Class IV laser light assembly. In this instance, the overall infrared energy photo-biotherapy medical treatment device or system comprises a head assembly, a coupling device and the Class IV laser light assembly. A liquid light guide is utilized to provide light delivery and connects the head assembly to the coupling device which, in turn, is connected to the Class IV laser light assembly via a plurality of silica optical fibers. In this manner, the liquid light guide, coupling device and plurality of optical fibers interconnects the head assembly with the Class IV laser light assembly. Optical lens are incorporated in both the head assembly and the coupling device permitting the laser light produced by the Class IV laser light assembly and transmitted via the plurality of optical fibers, coupling device and liquid light guide to be focused so as to penetrate into a living organism.

### BRIEF DESCRIPTION OF THE DRAWINGS

FIG. 1 is a perspective view of the overall infrared energy photo-biotherapy medical treatment device of the present invention.

FIG. 2 is a perspective view of the head assembly, liquid light guide, coupling device, optical fibers and laser light assembly utilized by the infrared energy photo-biotherapy medical treatment device illustrated in FIG. 1.

FIG. 3 is a perspective view of the head assembly shown in FIG. 2.

FIG. 4 is an elevational view of the head assembly shown in FIGS. 2 and 3.

FIG. 4A is a cross-sectional view of the head assembly taken across section-indicating lines 4A-4A in FIG. 4.

FIG. 5 is a perspective view of the head assembly shown in FIGS. 2 and 3 and illustrates the illumination “spot” produced thereby.

FIG. 6 is a perspective view of the coupling device shown in FIG. 2.

FIG. 7 is an elevational view of the coupling device shown in FIGS. 2 and 6.

FIG. 7A is a cross-sectional view of the coupling device taken across section-indicating lines 7A-7A in FIG. 7.

FIG. 7B is a cross-sectional view of the coupling device showing the optical fiber ferrule member in the removed position from the coupling device.

FIG. 7C is a cross-sectional view of the coupling device showing the optical fiber ferrule member inserted into the coupling device.

FIG. 8 is a perspective view of the Class IV laser light assembly utilized by the infrared energy photo-biotherapy medical treatment device of the present invention.

FIG. 9 is a perspective view of one of the Class IV laser light sources utilized by the infrared energy photo-biotherapy medical treatment device of the present invention.

FIG. 10 is an exploded view of the Class IV laser light source shown in FIG. 9.

FIG. 11 is a top plan view of the mounting plate assembly utilized by the Class IV laser light source shown in FIG. 9.

FIG. 11A is a cross-sectional view of the mounting plate assembly taken across section-indicating lines 11A-11A in FIG. 11.

FIG. 12 is a top plan view of the mounting plate assembly utilized by the Class IV laser light source shown in FIG. 9.

### DESCRIPTION OF THE PREFERRED EMBODIMENT

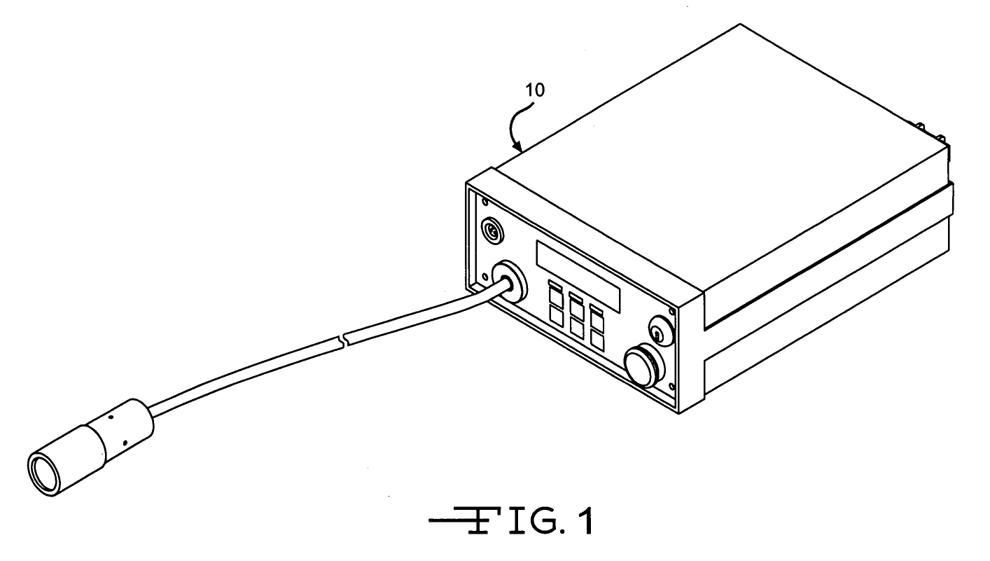

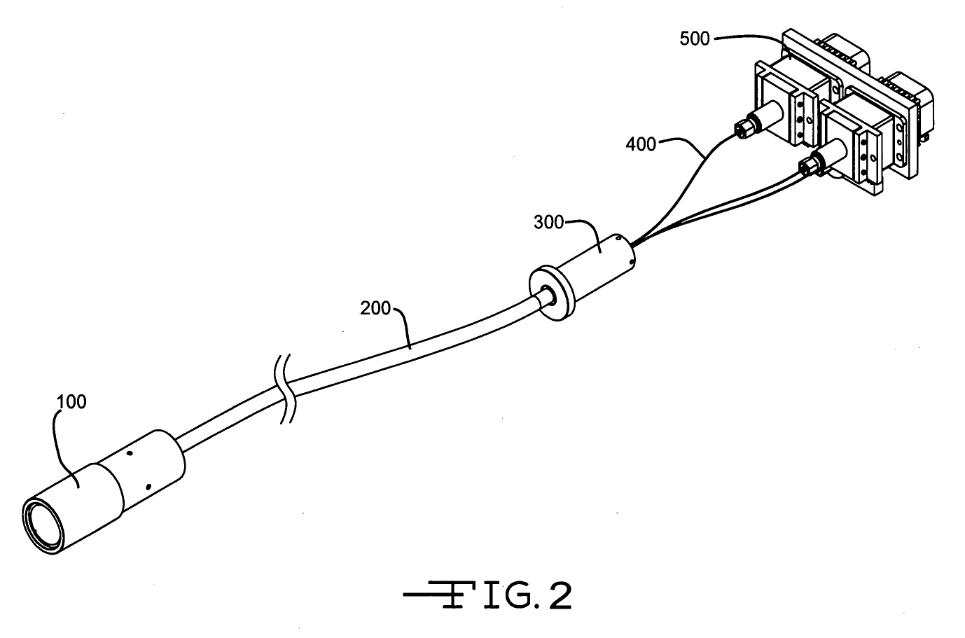

Referring now to the drawings where the illustrations are for the purpose of describing the preferred embodiment of the present invention and are not intended to limit the invention described herein, FIG. 1 is a perspective view of the overall infrared energy photo-biotherapy medical treatment device 10 of the present invention. As shown in FIG. 2, the medical treatment device 10 of the present invention is comprised of a head assembly 100, a liquid light guide 200, an optical fiber coupler 300 and a plurality of silica optical fibers 400, which is utilized to interconnect the coupler 300 to a Class IV laser light assembly 500. The liquid light guide 200 interconnects the head assembly 100 with the coupler 300.

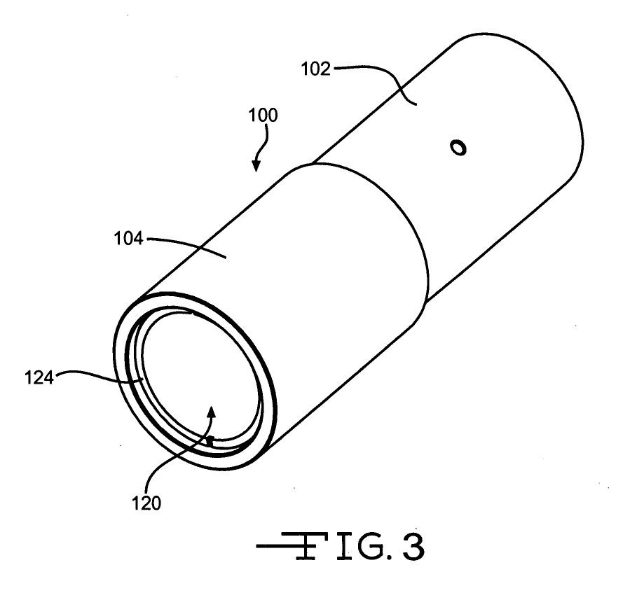

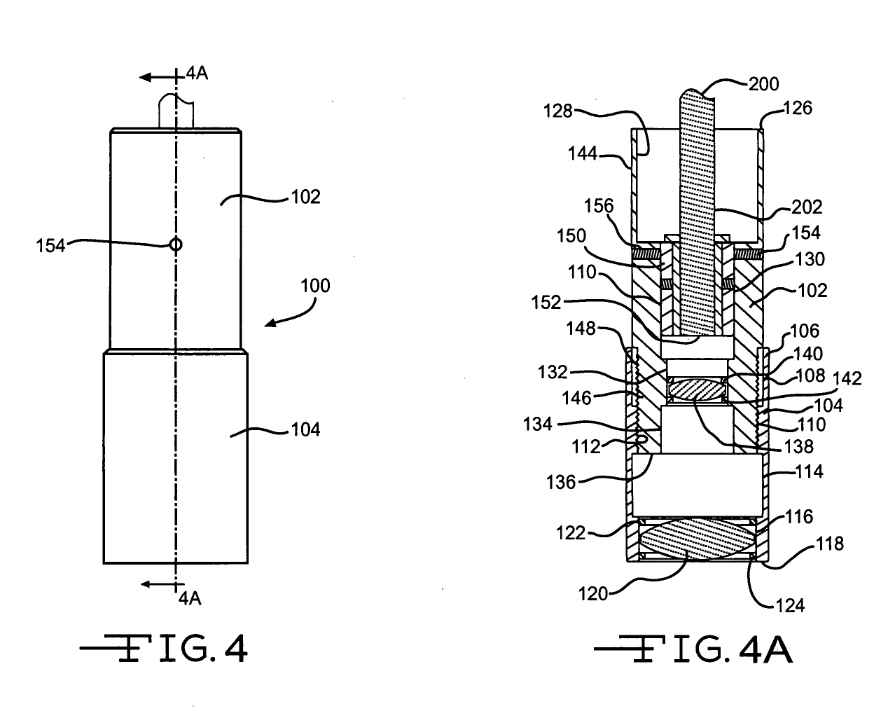

The head assembly 100, as shown in FIGS. 3, 4, 4A and 5, is comprised of an inner housing 102 and an outer housing 104, both of which are generally cylindrical in configuration. A portion of inner housing 102 is received within outer housing 104. End 106 of outer housing 104 has a blind bore 108 therein which terminates in a first bore 110 having a diameter slightly less than the diameter of blind bore 108. The surface of first bore 110 is provided with female threads 112 therein. First bore 110 terminates in a second bore 114 that has a diameter that approximates the diameter of blind bore 108. Second bore 114 terminates in a third bore 116 that terminates in opposite end 118 of outer housing 104. The diameter of third bore 116 is slightly less than the diameter of second bore 114. An optical lens 120 is received within third bore 116 and retaining rings 122, 124 are positioned on opposite sides of optical lens 120 to retain same in third bore 116.

End 126 of inner housing 102 has a blind bore 128 therein which terminates in a first bore 130 having a diameter less than the diameter of blind bore 128. First bore 130 terminates in a second bore 132 having a diameter slightly less than the diameter of first bore 130. Second bore 132 terminates in a third bore 134 that terminates in opposite end 136 of inner housing 102. The diameter of third bore 134 is greater than the diameter of second bore 132 and approximates the diameter of first bore 130. An optical lens 138 is received within second bore 132 and retaining rings 140, 142 are positioned on opposite sides of optical lens 138 to retain same in second bore 132. The outer surface of male housing 102 is comprised of a first circumferential portion 144 and a second circumferential portion 146 that has male threads 148 on a portion of the outer surface thereof. End 136 of inner housing 102 is received within end 106 of outer housing 104 and threadingly engages same through male threads 148 on the outer surface of second circumferential portion 146 of inner housing 102 and female threads 112 on first bore 110 of outer housing 104.

A liquid light guide ferrule 150 is received within bore 130 in inner housing 102 and is positioned therein such that its light emitting end 152 faces optical lens 138. Liquid light guide ferrule 150 is retained within bore 130 by set screws 154 which are oppositely disposed to one another and are received within threaded bores 156 in male housing 102.

The liquid light guide 200 is comprised of a plastic tube that is covered by a protective spiral of aluminum wire and a PVC jacket. The plastic tube is filled with a transparent, anaerobic, non-toxic fluid that facilitates the transmission of near infrared light. The tube is sealed at each of its oppositely disposed ends with a fused silica or glass window and is protected by an interlocking steel sheathing. One end 202 of the liquid light guide 200 is received within liquid light guide ferrule 150 that is within bore 130 in male housing 102.

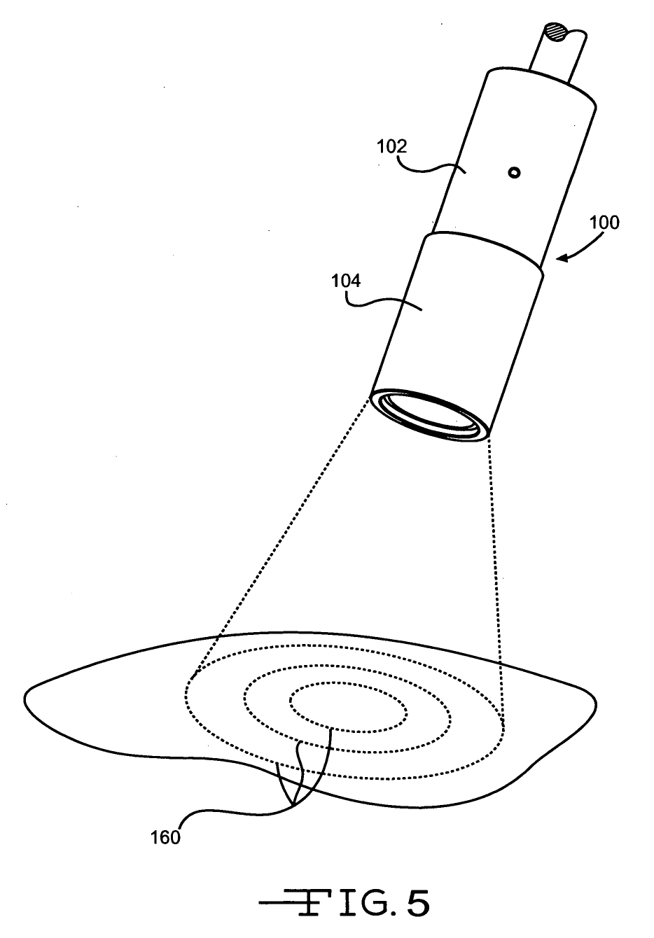

Referring now to FIG. 5, a perspective view of the head assembly 100 is shown and illustrates the size of the illumination “spot” 160 that can be produced thereby. The size of the illumination “spot” 160 can be varied by threadably advancing and or retracting the male housing 102 within the female housing 104. For example, when the inner housing 102 rotated clockwise within the outer housing 104, i.e., the inner housing 102 is threadably advanced into the outer housing 104, the size of the illumination “spot” 160 increases. Conversely, when the inner housing 102 is rotated counterclockwise with respect to the outer housing 104, i.e., the inner housing 102 is threadably retracted from the outer housing 104, the size of the illumination “spot” 160 decreases. It is also possible to utilize a fixed spot size wherein the head assembly 100 comprising the inner housing 102 and the outer housing 104 are molded as a unit preventing the inner housing 102 from being threadably advanced or retracted within the outer housing 104.

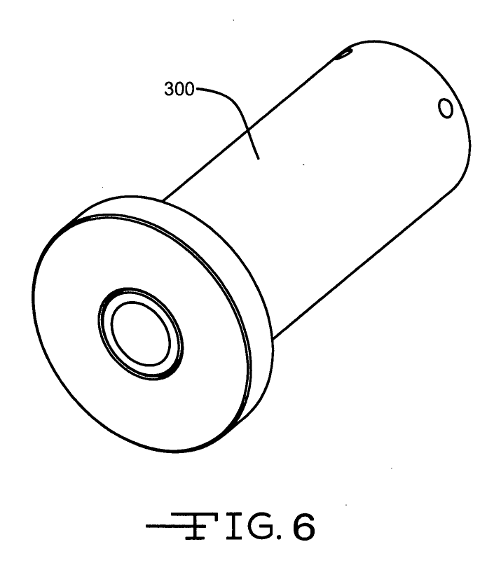

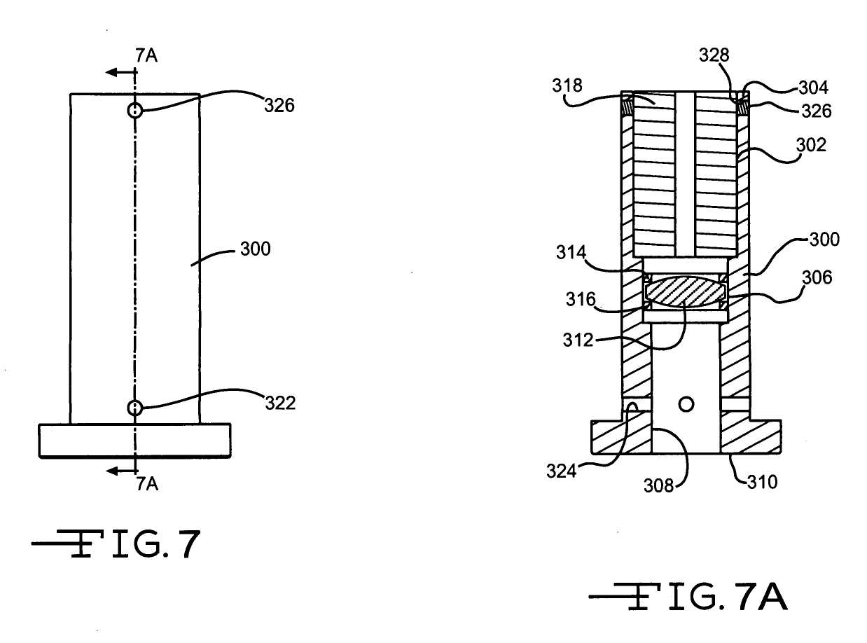

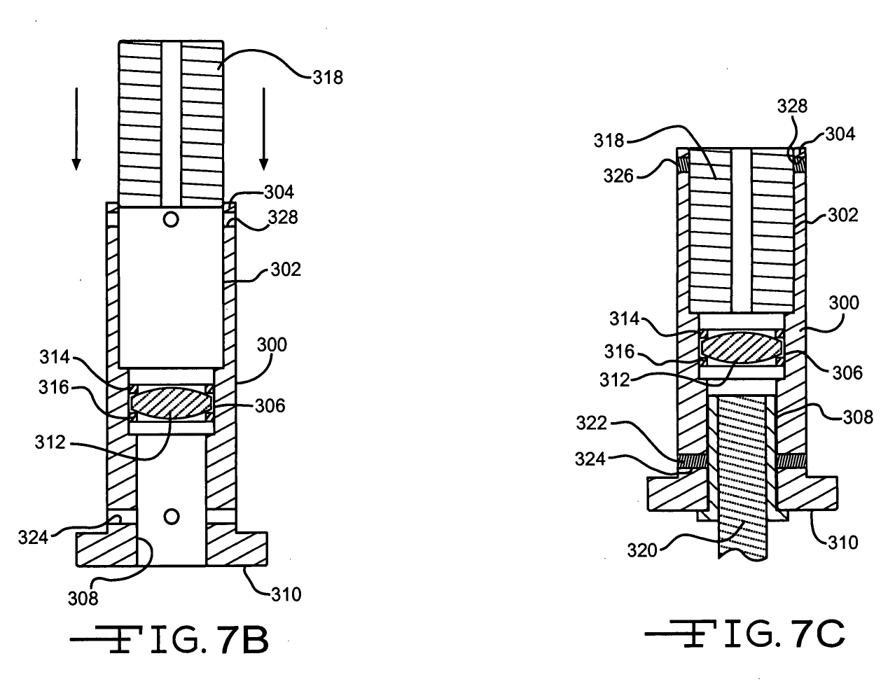

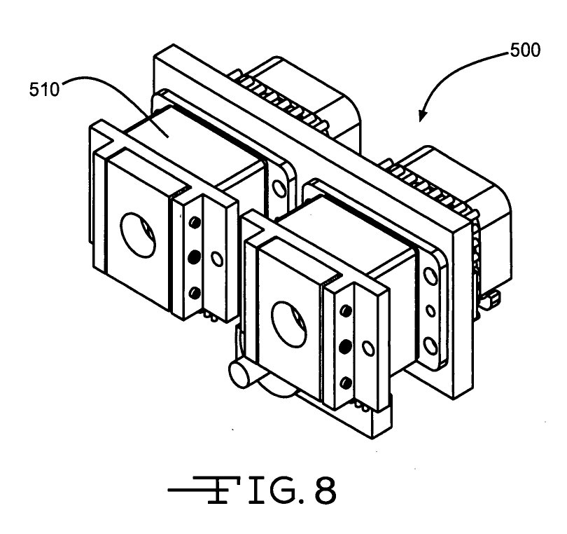

Referring now to FIG. 6, a perspective view of the optical fiber coupler 300 is illustrated. As shown in FIGS. 7, 7A, 7B and 7C, the coupler 300 is typically cylindrical in configuration and has a blind bore 302 in end 304 thereof. Blind bore 302 terminates in a first bore 306 having a diameter less than the diameter of blind bore 302. First bore 306 terminates in second bore 308 provided in opposite end 310 of coupler 300. An optical lens 312 is received within first bore 306 and retaining rings 314, 316 are positioned on opposite sides of optical lens 312 to retain same in first bore 306. An optical fiber ferrule 318 is received within blind bore 302 of coupler 300. The other end 320 of the liquid light guide 200 is received in second bore 308 and is retained therein by oppositely disposed set screws 322 that are threadably received within threaded bores 324 in coupler 300 adjacent end 310 thereof, as shown in FIG. 7C. It should be noted that FIGS. 7B and 7C illustrate the lateral positioning of optical fiber ferrule 318 in blind bore 302 of coupler 300 to achieve the proper focal distance from the emission end of the optical fiber ferrule 318, through the optical lens 312, and into the end 320 of the liquid light guide 200. After the optical fiber ferrule 318 has been laterally positioned in blind bore 302 in coupler 300, the position of the optical fiber ferrule 318 is maintained by oppositely disposed set screws 326 that are received within threaded bores 328 in coupler 300.

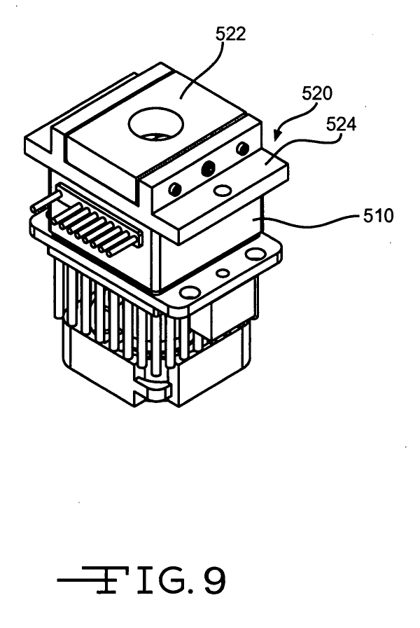

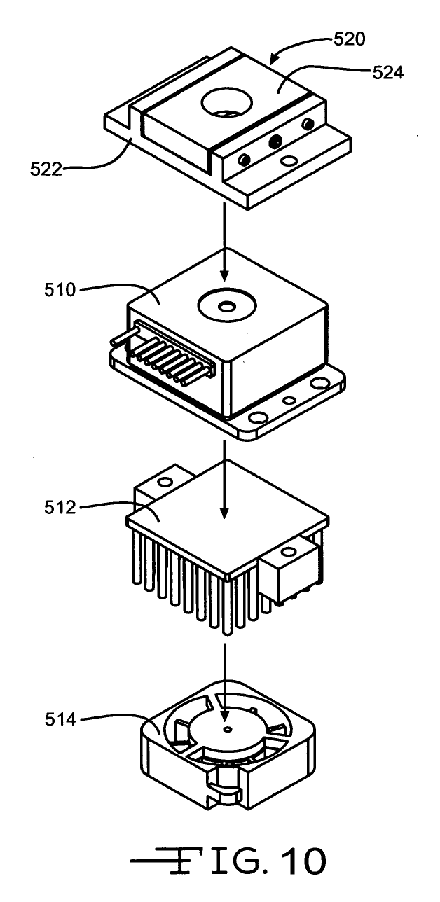

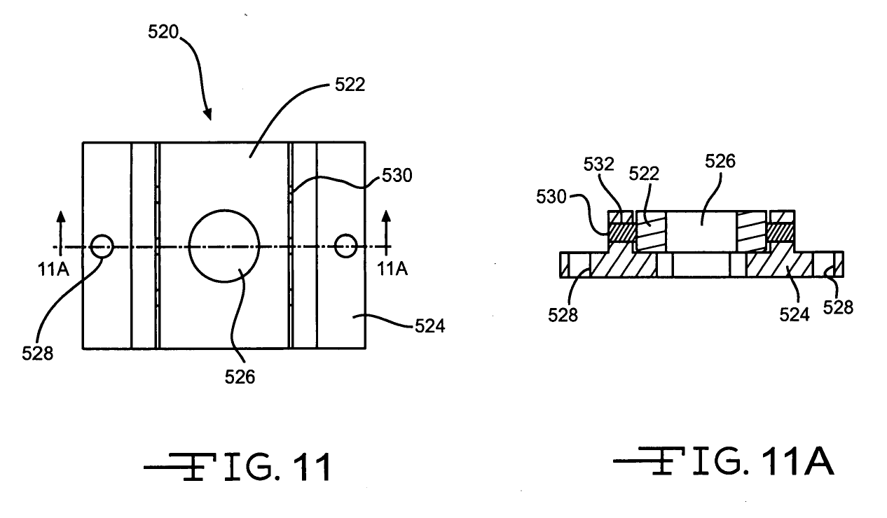

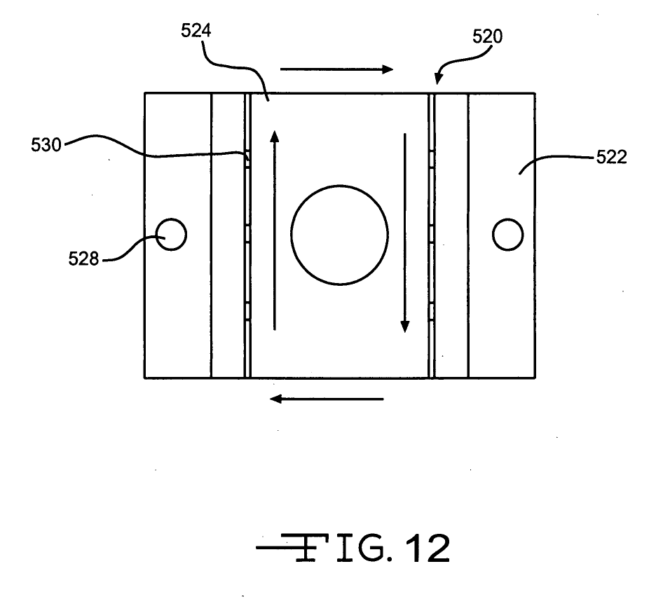

Referring now to FIGS. 8 and 9, perspective views of the Class IV laser light assembly 500 and a laser light source 510 within the assembly 500, respectively, are illustrated. Similarly, an exploded view of laser light source 510 is illustrated in FIG. 10. Laser light source 510 includes a mounting plate assembly 520 comprised of a top plate 522 and a bottom plate 524. Referring now to FIGS. 11, 11A and 12, top plate 522 has an aperture 526 therein and is laterally movable within bottom plate 524. Apertures 528 are provided in bottom plate 524 permitting bottom plate 524 to be affixed to the emission surface of the laser light source 510. Lateral movement of the top plate 522 with respect to the bottom plate 524 optimizes the output power of the laser light source 510 into the plurality of silica optical fibers 400. The position of top plate 522 within bottom plate 524 is maintained by oppositely disposed set screws 530 that are received within threaded bores 532 in bottom plate 524.

To investigate the efficacy of low-level exposure to 980 nm laser light produced by the infrared energy photo-biotherapy medical treatment device 10 of the present invention, cell growth rates following wound induction using an in-vitro model of wound healing were investigated. A small pipette was used to mechanically induce a wound in fibroblast cell cultures, which were then imaged at specific time intervals following wound induction and exposed to various doses of laser light. Results indicate that exposure to low and medium intensity laser light significantly accelerated cell growth and that high intensity laser light negated the beneficial effects of laser light exposure on cell growth. Further experimentation demonstrated that cell growth was accelerated over a wide range of exposure durations using medium intensity laser light with no marked reduction in cell growth at the longest exposure duration. The test results confirm clinical observations that low-level exposure to 980 nm laser light can accelerate the healing of superficial wounds.

Regarding the testing procedure utilized, a range of exposure doses was investigated by varying the output power of the laser light source over a fixed exposure duration, or by varying the exposure duration at a fixed output power of the laser light source. In the first experiment, the output power of the laser light source was varied from 1.5-7.5 watts to produce an exposure level of 2.6-120 mw/cm² over a two minute exposure interval, resulting in exposure doses from 3.1-15.4 J/cm². It was found that regardless of exposure levels, significant cell recovery was observed within three hours of wound induction, however, exposure to moderate exposure levels (26-97 mw/cm²) appeared to enhance cell growth at all time intervals relative to control experiments in which no laser light exposure was applied. The results also showed that the beneficial effects of laser light exposure were negated by over-exposure since fibroblasts exposed to exposure levels of 120 mw/cm² for two-minute intervals did not show any significant increase in cell growth rates relative to control experiments.

In the second experiment, exposure durations were varied from 20 seconds-15 minutes at a substantially constant output power of 4.5 watts of the laser light source to produce an exposure level of 73 mw/cm², resulting in exposure doses from 1.5-66 J/cm². As with changes in exposure levels, significant cell recovery was observed within three hours of wound induction regardless of exposure duration, and a wide range of exposure durations appeared to enhance cell growth at all time intervals relative to control experiments in which no laser light exposure was applied.

The foregoing test results confirm the clinical observation that low-level exposure to 980 nm of laser light can accelerate cell growth in a wound healing model. Because the test measurements were obtained from an in-vitro culture model, the results also suggest that the mechanisms involved in the acceleration of cell growth following laser light exposure are cellular or molecular in nature. The results also demonstrate the importance of appropriate supervision of laser light exposure. In particular, the average cell growth rate formed a non-monotonic function of laser light exposure levels and exposure doses with peak growth rates at moderate exposures and reduced benefit at higher exposure intensities and doses.

Certain modifications and improvements will occur to those skilled in the art upon reading the foregoing. It is understood that all such modifications and improvements have been deleted herein for the sake of conciseness and readability, but are properly within the scope of the following claims.

We claim:

1) An infrared light transmission system comprising a head member, a coupling member, a light source, means for interconnecting said head member with said coupling member and means for interconnecting said coupling member with said light source.

2) The system as defined in claim 1 wherein said head member comprises a housing and a plurality of optical lenses received within said housing.

3) The system as defined in claim 2 wherein said plurality of optical lenses within said housing comprises a first optical lens and a second optical lens.

4) The system as defined in claim 3 wherein said first optical lens and said second optical lens are in a spaced-apart relationship within said housing.

5) The system as defined in claim 4 wherein the lateral distance between said first optical lens and said second optical lens within said housing can be varied.

6) The system as defined in claim 4 wherein the lateral distance between said first optical lens and said second optical lens within said housing is substantially fixed.

7) The system as defined in claim 4 wherein said housing comprises a first housing member and a second housing member, said first optical lens being received within said first housing member and said second optical lens being received within said second housing member.

8) The system as defined in claim 7 further including means for adjustably receiving at least a portion of said second housing member within said first housing member.

9) The system as defined in claim 8 wherein said adjustable receiving means comprises a plurality of threads on said first housing member and a plurality of mating threads on said second housing member.

10) The system as defined in claim 1 wherein said coupling member comprises a housing and an optical lens received within said housing.

11) The system as defined in claim 1 wherein said light source is a Class IV laser light assembly.

12) The system as defined in claim 1 wherein said means for interconnecting said head member with said coupling member comprises a liquid light guide member.

13) The system as defined in claim 1 wherein said means for interconnecting said coupling member with said light source comprises a plurality of optical fibers.

14) The system as defined in claim 1 further including means for varying the output power of said light source.

15) The system as defined in claim 14 wherein said output power varying means comprises a plate assembly operably attached to said light source, said plate assembly comprising a first plate member and a second plate member, said first plate member being operably attached to the emission surface of said light source and having an aperture therein, said second plate member being slidingly movable with respect to said first plate member and having an aperture therein, the orientation of said aperture in said second plate member with respect to said aperture in said first plate member regulating the amount of infrared energy delivered by said light source to said head member through said means interconnecting said coupling member with said light source, said coupling member, and said means interconnecting said head member with said coupling member.

16) A method of enhancing wound healing by utilizing a light source, a liquid light guide and a coupling member interconnecting said light source and said liquid light guide by irradiating said wound with light produced by said light source and by varying the output power produced by said light source over a substantially fixed exposure duration.

17) A method of enhancing wound healing by utilizing a light source, a liquid light guide and a coupling member interconnecting said light source and said liquid light guide by irradiating said wound with light produced by said light source and by varying the exposure duration while maintaining the output power produced by said light source substantially constant.
